(19) United States
(12) Patent Application Publication
(10) Pub. No.: US 2026/0145008 A1
(43) Pub. Date: May 28, 2026

(54) TIME-SERIES SIGNAL PREDICTION DEVICE, RADIATION TREATMENT DEVICE, TIME-SERIES SIGNAL PREDICTION METHOD, AND STORAGE MEDIUM

(71) Applicants: Toshiba Energy Systems & Solutions Corporation, Kawasaki-shi (JP); National Institutes for Quantum Science and Technology, Chiba-shi (JP)

(72) Inventors: Ryusuke HIRAI, Meguro Tokyo (JP); Shinichiro MORI, Chiba-shi (JP); Yasuhiro SOEKAWA, Shinagawa Tokyo (JP)

(73) Assignees: Toshiba Energy Systems & Solutions Corporation, Kawasaki-shi (JP); National Institutes for Quantum Science and Technology, Chiba-shi (JP)

(21) Appl. No.: 19/406,264

(22) Filed: Dec. 2, 2025

Related U.S. Application Data
(63) Continuation of application No. PCT/JP2024/020754, filed on Jun. 6, 2024.

(30) Foreign Application Priority Data
Jun. 21, 2023 (JP) 2023-101589

(51) Int. Cl. A61N 5/10

(52) U.S. Cl. CPC A61N 5/1068; A61N 5/1049; A61N 5/107; A61N 2005/1061

(57) ABSTRACT

A time-series signal prediction device according to an embodiment includes a time-series signal acquirer, a block converter, a feature vector acquirer, and a feature vector converter. The time-series signal acquirer acquires a time-series signal associated with a patient's biological information. The block converter divides the time-series signal into sub signals at predetermined intervals and converts the sub signals to block signals of a time series which is shorter than a time series of the time-series signal. The feature vector acquirer acquires, from the block signals, a feature vector indicating a time-series signal at a time subsequent to the time at which the time-series signal has been acquired. The feature vector converter converts the feature vector to a prediction signal associated with the patient's biological information.

### CROSS-REFERENCE TO RELATED APPLICATION

This application is based upon and claims the benefit of priority from Japanese Patent Application No. 2023-101589, filed Jun. 21, 2023 and PCT/JP2024/020754, filed Jun. 6, 2024; the entire contents all of which are incorporated herein by reference.

### FIELD

An embodiment of the present invention relates to a time-series signal prediction device, a radiation treatment device, a time-series signal prediction method, and a storage medium.

### BACKGROUND

Radiation treatment is a treatment method of destroying a patient's lesion by irradiating the lesion with radiation. When a position of the lesion is not accurately irradiated with radiation, normal tissue is also destroyed. Accordingly, a lesion position in a patient's body is three-dimensionally ascertained by performing CT imaging in advance, and an irradiation direction or an irradiation intensity is planned such that irradiation of normal tissue is reduced.

In radiation treatment, a patient's position at the time of treatment needs to be aligned with that in a treatment planning stage in order to perform irradiation with radiation according to a plan. In order to align a position of a lesion, a bone, or the like in a patient's body with that in the treatment planning stage, there is a method of comparing a fluoroscopic image in the body of a patient laid in a bed immediately before treatment in a treatment room with a digitally reconstructed radiograph (DRR) obtained by virtually reconstructing a fluoroscopic image from three-dimensional CT images captured in the treatment planning stage to acquire a misalignment of the patient between the images and moving the bed on the basis of the misalignment. The misalignment of the position of the patient is acquired by searching for a position in a CT image from which a DRR most similar to the fluoroscopic image is reconstructed. A plurality of methods of automating this search using a computer have been proposed, and an employee for radiation treatment needs to ascertain the search result by comparing the fluoroscopic image with the DRR.

A respiratory-gated irradiation method of identifying a position of a tumor under irradiation when the tumor which is a lesion of a patient is located in an organ moving with movement of respiration or a heartbeat such as the lungs or the liver and irradiating the tumor with a treatment beam when the identified position matches a position determined by treatment planning is known. At this time, as a method of estimating a position of a tumor, there is a method of sensing a motion of a thoracoabdominal body surface which expands/contracts with respiration and estimating a position of a tumor according to the sensor value. Alternatively, there is also a method of capturing a fluoroscopic video of a patient under irradiation and tracking a tumor position in the fluoroscopic video. In the following description, a signal which can be acquired from the motion of the body surface or a trajectory of the tumor position is referred to as a “time-series signal.” Unnecessary irradiation of a patient with a treatment beam can be reduced by controlling an irradiation timing of the treatment beam in this way, and it is necessary to predict the tumor position due to a response time of a device such as a calculation time required for estimating the tumor position.

For example, in a method disclosed in “Towards real-time respiratory motion prediction based on long short-term memory neural networks,” Phys Med Biol. 2019 Apr. 10; 64(8): 085010, a signal value at a predetermined time is predicted by training a long-short term memory (LSTM) which is a recurrent network using time-series signals of a plurality of patients. In a method disclosed in“Respiratory Prediction Based on Multi-Scale Temporal Convolutional Network for Tracking Thoracic Tumor Movement,” Front. Oncol., 27 May 2023, unlike the method disclosed in “Towards real-time respiratory motion prediction based on long short-term memory neural networks,” Phys Med Biol. 2019 Apr. 10; 64(8): 085010, a convolutional neural network (CNN) which is a feedforward network is used. The disclosed technique is a method of predicting a signal value at a future time (T+M, M>0) from a time-series signal up to a certain time (T). In this method, it can be expected to improve the accuracy as a length of a time-series signal to be input is increased, but since a calculation time is also increased therewith, the method may not be appropriate for prediction. When waveform signals from time T+1 to time T+M are learned, an amount of information used for learning increases in comparison with a case in which only the signal value at time T+M is used, and thus it can be expected to improve the prediction accuracy.

An objective of the invention is to provide a time-series signal prediction device, a radiation treatment device, a time-series signal prediction method, and a storage medium that can predict a time-series signal associated with a patient's biological information at a high speed and with high accuracy.

A time-series signal prediction device according to an embodiment includes a time-series signal acquiring unit, a block conversion unit, a feature vector acquiring unit, and a feature vector conversion unit. The time-series signal acquiring unit acquires a time-series signal associated with a patient's biological information. The block conversion unit divides the time-series signal into sub signals at predetermined intervals and converts the sub signals to block signals of a time series which is shorter than a time series of the time-series signal. The feature vector acquiring unit acquires, from the block signals, a feature vector indicating a time-series signal at a time subsequent to the time at which the time-series signal has been acquired. The feature vector conversion unit converts the feature vector to a prediction signal associated with the patient's biological information.

According to the present invention, it is possible to provide a time-series signal prediction device, a radiation treatment device, a time-series signal prediction method, and a storage medium that can predict a time-series signal associated with a patient's biological information at a high speed and with high accuracy.

### BRIEF DESCRIPTION OF DRAWINGS

FIG. 1 is a block diagram schematically illustrating a configuration of a treatment system including a time-series signal prediction device according to a first embodiment.

FIG. 2 is a block diagram schematically illustrating a configuration of a time-series signal prediction device 100 according to the first embodiment.

FIG. 3 is a diagram illustrating an example of a waveform signal that is acquired by a time-series signal acquiring unit 110.

FIG. 4 is a diagram illustrating an example in which a time-series signal is converted to block signals by a block conversion unit 120.

FIG. 5 is a diagram illustrating another example in which a time-series signal is converted to block signals by the block conversion unit 120.

FIG. 6 is a diagram illustrating an example in which a feature vector is acquired by a feature vector acquiring unit 130.

FIG. 7 is a diagram illustrating another example in which a feature vector is acquired by the feature vector acquiring unit 130.

FIG. 8 is a flowchart illustrating an example of a process flow that is performed by the time-series signal prediction device 100 according to the first embodiment.

FIG. 9 is a block diagram schematically illustrating a configuration of a time-series signal prediction device 200 according to a second embodiment.

FIG. 10 is a flowchart illustrating an example of a process flow that is performed by the time-series signal prediction device 200 according to the second embodiment.

FIG. 11 is a block diagram schematically illustrating a configuration of a time-series signal prediction device 300 according to a third embodiment.

FIG. 12 is a flowchart illustrating an example of a process flow that is performed by the time-series signal prediction device 300 according to the third embodiment.

### DETAILED DESCRIPTION

Hereinafter, a time-series signal prediction device, a radiation treatment device, a time-series signal prediction method, and a storage medium according to embodiments will be described with reference to the accompanying drawings.

### First Embodiment

### (Entire Configuration)

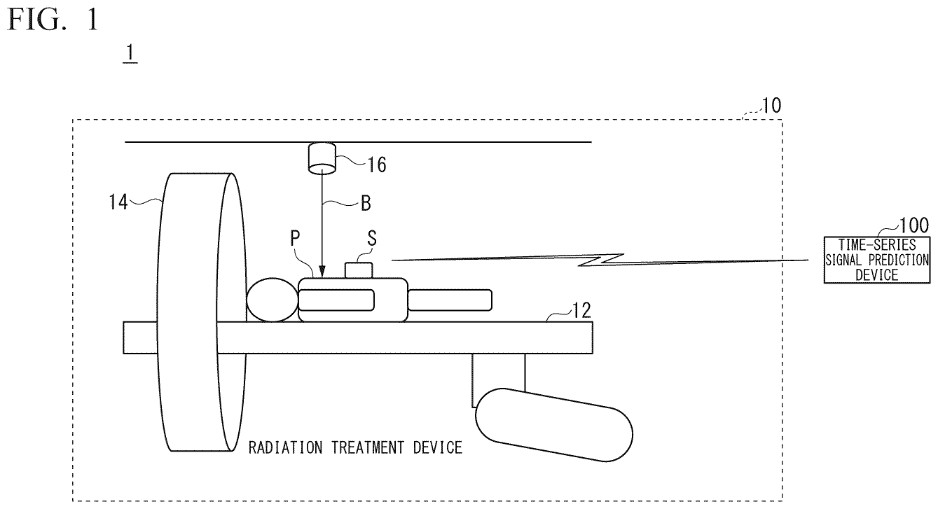

FIG. 1 is a block diagram schematically illustrating a configuration of a treatment system including a time-series signal prediction device according to a first embodiment. A treatment system 1 includes, for example, a treatment device 10, a sensor S, and a time-series signal prediction device 100. The treatment device 10 includes, for example, a bed 12, a computed tomography (CT) device 14 (hereinafter referred to as a “CT imaging device 14”), and a treatment beam emission gate 16.

The bed 12 is a movable treatment table that fixes a test subject (patient) P who is subjected to radiation treatment, for example, in a state in which the test subject is lying thereon using a fixture or the like. The bed 12 moves in a state in which a patient P is fixed in the CT imaging device 14 of a ring shape including an opening under the control of a bed control unit (not illustrated). The bed control unit controls a translation mechanism and a rotation mechanism which are provided in the bed 12 such that a direction in which the patient P fixed to the bed 12 is irradiated with a treatment beam B is changed according to a movement amount signal. The translation mechanism can drive the bed 12 in three-axis directions, and the rotation mechanism can drive the bed 12 around three axes. Accordingly, the bed control unit controls the translation mechanism and the rotation mechanism of the bed 12, for example, such that the bed 12 moves in six decrees of freedom. The number of degrees of freedom in which the bed control unit controls the bed 12 may not be six and may be less than six (for example, four degrees of freedom) or may be greater than six (for example, eight degrees of freedom).

The CT imaging device 14 is an imaging device for performing three-dimensional computed tomography. In the CT imaging device 14, a plurality of radiation sources are provided inside of a ring-shaped opening, and radiation for seeing through the body of the patient P is emitted from each radiation source. That is, the CT imaging device 14 emits radiations from a plurality of positions around the patient P. The radiation emitted from each radiation source in the CT imaging device 14 is, for example, X-rays. The CT imaging device 14 detects radiations emitted from the corresponding radiation sources, passing through the body of the patient P, and reaching a plurality of radiation detectors provided inside of the ring-shaped opening using the plurality of radiation detectors. The CT imaging device 14 generates a CT image of the internal body of the patient P on the basis of a magnitude of energy of radiation detected by each radiation detector. The CT image of the patient P generated by the CT imaging device 14 is a three-dimensional digital image in which the magnitude of energy of radiation is represented by a digital value. Three-dimensional imaging of the internal body of the patient P in the CT imaging device 14, that is, emission of radiation from each radiation source or generation of a CT image based on radiation detected by each radiation detector, is controlled, for example, by an imaging control unit (not illustrated).

The treatment beam emission gate 16 emits radiation for destroying a tumor (a lesion) which is a treatment target region in the body of the patient P as a treatment beam B. Examples of the treatment beam B include an X-ray, a γ-ray, an electron beam, a proton beam, a neutron beam, and a baryon beam. The patient P (more specifically, the tumor in the body of the patient P) is irradiated with the treatment beam B linearly from the treatment beam emission gate 16. Emission of the treatment beam B from the treatment beam emission gate 16 is controlled, for example, by a treatment beam irradiation control unit (not illustrated).

In this way, radiation treatment is performed by irradiating a tumor in the body of the patient P with a treatment beam B from the treatment beam emission gate 16, but when the tumor which is a lesion of the patient P is located in an organ moving with movement of respiration or a heartbeat such as the lungs or the liver, emission of an X-ray from the CT imaging device 14 or emission of a treatment beam B from the treatment beam emission gate 16 needs to be performed at the timing at which the tumor is included in the irradiation range of the treatment beam B. However, in the related art, even when a tumor is located outside of an irradiation range of a treatment beam B, an X-ray or a treatment beam B may be emitted to cause unnecessary exposure of the patient P, for example, due to a gap of a machine response. In consideration of these circumstances, the present invention enables predicting a timing at which a tumor is located inside of the irradiation range of a treatment beam B at a high speed and with high accuracy by using a sensor S and a time-series signal prediction device 100.

The sensor S continuously senses a motion of a thoracoabdominal body surface expanding/contracting with respiration of the patient P and outputs a one-dimensional waveform signal with the acquired sensor value as the amplitude to the time-series signal prediction device 100. The sensor S may be a contact type sensor which is located on the top surface of the abdomen of the patient P laid on the bed 12 or may be a noncontact type sensor for measuring a distance to the chest and the abdomen using a laser.

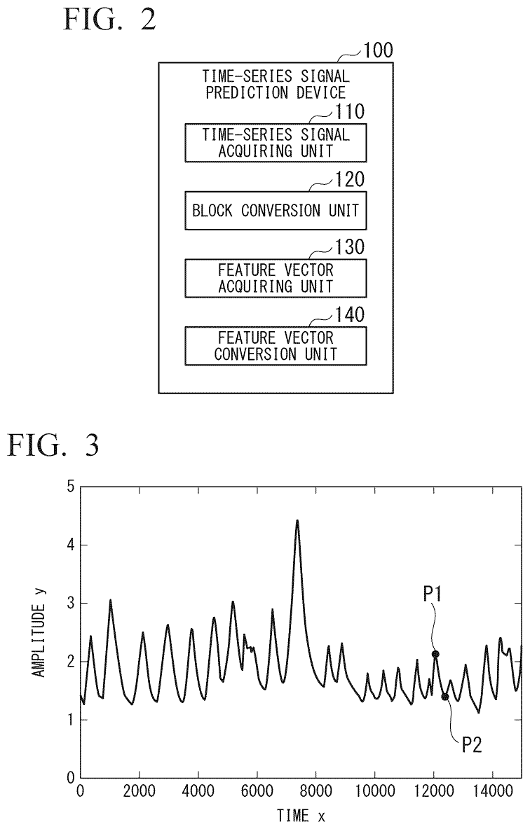

FIG. 2 is a block diagram schematically illustrating a configuration of a time-series signal prediction device 100 according to the first embodiment. The time-series signal prediction device 100 includes, for example, a time-series signal acquiring unit 110, a block conversion unit 120, a feature vector acquiring unit 130, and a feature vector conversion unit 140. Some or all of the constituents of the time-series signal prediction device 100 are realized, for example, by causing a hardware processor such as a central processing unit (CPU) to execute a program (software). Some or all of the constituents may be realized by hardware (a circuit unit which includes circuitry) such as a large scale integration (LSI) circuit, an application-specific integrated circuit (ASIC), a field-programmable gate array (FPGA), or a graphics processing unit (GPU) or may be cooperatively realized by software and hardware. Some or all functions of the constituents may be realized by a dedicated LSI circuit. The program may be stored in a storage device (a storage device including a non-transitory storage medium) such as a read only memory (ROM), a random access memory (RAM), a hard disk drive (HDD), or a flash memory provided in the time-series signal prediction device 100 in advance or may be stored in a removable storage medium (a non-transitory storage medium) such as a DVD or a CD-ROM and installed in the HDD or the flash memory provided in the time-series signal prediction device 100 by setting the storage medium into a drive device of the time-series signal prediction device 100. The program may be downloaded from another computer device via a network and installed in the HDD or the flash memory of the time-series signal prediction device 100.

The time-series signal acquiring unit 110 acquires a time-series signal indicating a waveform from the sensor S, for example, by wireless communication. FIG. 3 is a diagram illustrating an example of a waveform signal acquired by the time-series signal acquiring unit 110. In the graph illustrated in FIG. 3, the horizontal axis represents time, and the vertical axis represents amplitude. In the waveform illustrated in FIG. 3, a peak P1 which is a mountain indicates a point at which inhalation of the patient P is maximized, and a peak P2 which is a valley indicates a point at which exhalation of the patient P is maximized. The time-series signal acquiring unit 110 outputs the acquired time-series signal to the block conversion unit 120.

For example, the time-series signal acquired by the time-series signal acquiring unit 110 may be a trajectory indicating a position of a tumor moving with respiration of the patient P. Here, the trajectory indicating a position of a tumor means a time-series signal indicating temporal change of a center position of the tumor in a fluoroscopic video of the patient P. In this case, the time-series signal acquiring unit 110 may acquire the time-series signal from the CT imaging device 14 instead of the sensor S.

For example, the time-series signal acquired by the time-series signal acquiring unit 110 may be a time-series signal indicating temporal change of the diaphragm of the patient P or may be a trajectory along which a marker placed in the vicinity of a tumor in the body of the patient P moves with respiration. The trajectory of the marker is a time-series signal indicating temporal change of a marker position in a fluoroscopic video of the patient. The positions of the tracking targets such as the tumor, the diaphragm, and the marker (hereinafter referred to as tracking targets) in a fluoroscopic video at each time are estimated, for example, by template matching using an image in which a tracking target appears before tracking as a template. Alternatively, for example, the position of the tracking target at each time is estimated on the basis of a feature quantity to which the image in which the tracking target appears is converted using a model which has been prepared in advance. The model at this time can be constructed by machine learning for learning a plurality of images in which a tracking target appears before tracking.

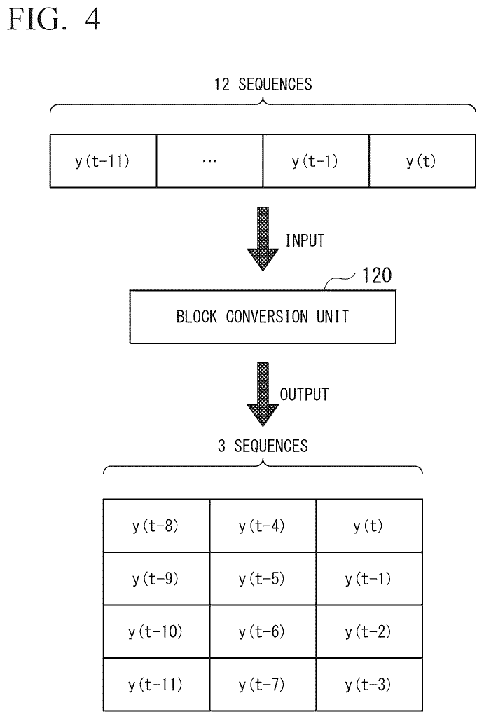

The block conversion unit 120 divides the time-series signal acquired by the time-series signal acquiring unit 110 into sub signals at predetermined intervals and converts the sub signals to block signals of a time series which is shorter than a time series of the time-series signal. FIG. 4 is a diagram illustrating an example in which a time-series signal is converted to block signals by the block conversion unit 120. FIG. 4 illustrates an example in which a time-series signal of 12 sequences including 12 signal values y(t−11) to y(t) from time t−11 to time t (that is, a current time) is converted to block signals of 3 sequences. The sub signals at this time are vector data including signal values y(t−3) to y(t) from time t−3 to time t, signal values y(t−7) to y(t−4) from time t−7 to t−4, and signal values y(t−11) to y(t−8) from time t−11 to time t−8.

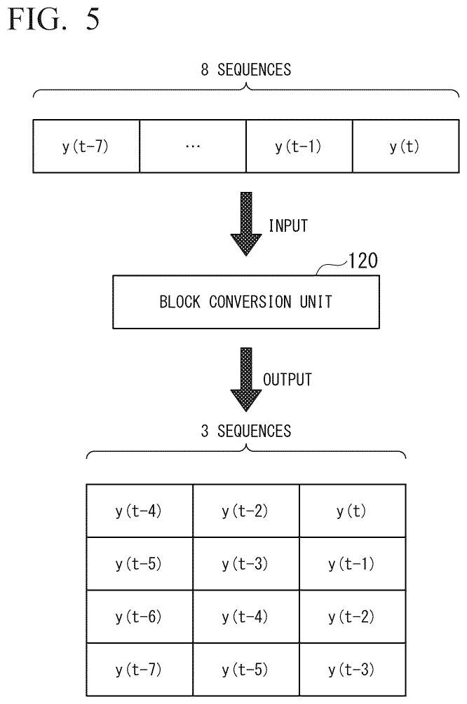

FIG. 5 is a diagram illustrating another example in which a time-series signal is converted to block signals by the block conversion unit 120. FIG. 5 illustrates an example in which signal values of 8 sequences including 8 signal values y(t−7) to y(t) from time t−7 to time t is converted to block signals of 3 sequences. The sub signals at this time are vector data including signal values y(t−3) to y(t) from time t−3 to time t, signal values y(t−5) to y(t−2) from time t−5 to t−2, and signal values y(t−7) to y(t−4) from time t−7 to time t−4. In this way, duplication of a signal value between sequences may be permitted without changing the order of the time series.

The feature vector acquiring unit 130 acquires a feature vector indicating a time-series signal at a time subsequent to the time at which the time-series signal has been acquired from the block signals on which conversion has been performed by the block conversion unit 120. More specifically, in order to predict a time-series signal at a future time from an input time-series signal, the feature vector acquiring unit 130 is constructed in advance as a predictor obtained by statistically modeling variation of a signal from collected time-series signals associated with biological information of a plurality of patients.

As a method of modeling the feature vector acquiring unit 130, for example, there is a method of constructing a state space model. Specifically, when a pair of two waveform values y(t) and y(t−δ) at time t and time t−δ are plotted in a two-dimensional coordinate system, a respiration waveform is periodic, and thus the plotted points move in an elliptical trajectory with time. These characteristics are expressed by a state space model. Since this model is a nonlinear model, an extended Kalman filter (EKF) obtained by approximating a Kalman filter to correspond to the state space model which is nonlinear is used. The feature vector acquiring unit 130 is constructed by optimizing parameters of the state space model using the collected time-series signals.

As another method of modeling the feature vector acquiring unit 130, for example, there is a method using a recurrent neural network. Here, the recurrent neural network is a neural network model that obtains an output by causing an internal state of the model to transition when input data of a plurality of sequences is sequentially input thereto. A representative example thereof is a long-short term memory (LSTM).

For example, a feedforward neural network is used as another method of modeling the feature vector acquiring unit 130. Here, the feedforward neural network is a neural network model that obtains an output when input data is input in batch without dividing the input data into sequences. For example, when the feedforward neural network is a convolutional neural network (CNN), processing is performed with block signals considered as two-dimensional data when block signals are input thereto. Alternatively, sequences of the block signals may be input as data of different channels to the network.

When these neural network models are used, the feature vector acquiring unit 130 is constructed by training the internal parameters of the model through deep learning using the collected time-series signals. At this time, the collected time-series signals are divided into a plurality of sub signals with duplication permitted, and each sub signal is divided into a training block signal to be input and a target feature vector. Data included in the target feature vector includes only signals at a time subsequent to the training block signal.

The training is to adjust the internal parameters such that a feature vector acquired as an output when a training block signal is input to the model approaches the target feature vector. A function of measuring a distance between the target feature vector and the feature vector is used as an error function. An optimization method such as an error back propagation method is used to minimize the error function.

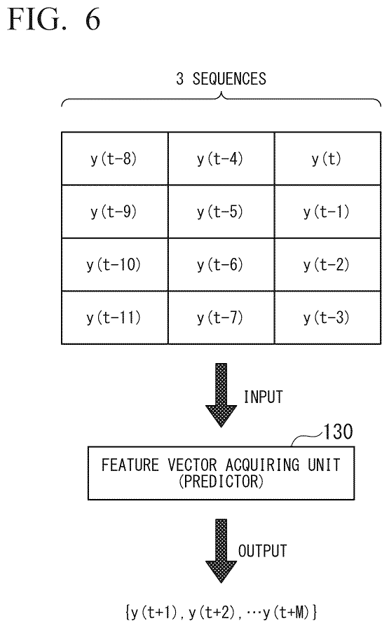

FIG. 6 is a diagram illustrating an example in which a feature vector is acquired by the feature vector acquiring unit 130. As illustrated in FIG. 6, the feature vector is, for example, a vector with amplitude values of the sub signals as elements. That is, when a signal value at time t+M (M>0, M is a natural number) is predicted in response to training block signals up to time t, the target feature vector is a vector with signal values y(t+1) to y(t+M) from time t+1 to time t+M as elements.

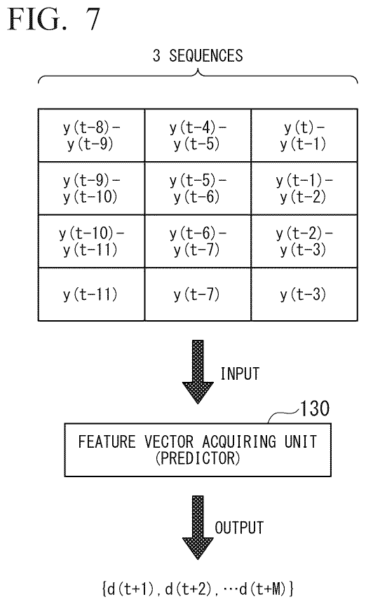

FIG. 7 is a diagram illustrating another example in which a feature vector is acquired by the feature vector acquiring unit 130. As illustrated in FIG. 7, a feature vector may be, for example, a vector with differences between amplitude values of the sub signals as elements, That is, when a signal value y(t+M) at time t+M (M>0, M is a natural number) is predicted in response to training block signals up to time t, the target feature vector is a vector with the training block signal at time t as a start point and with differences d(t+1) to d(t+M) between signal values from time t+1 to time t+M as elements.

The feature vector conversion unit 140 converts the feature vector acquired by the feature vector acquiring unit 130 to a prediction signal associated with biological information of the patient P. More specifically, when the feature vector illustrated in FIG. 6 is acquired by the feature vector acquiring unit 130, the feature vector conversion unit 140 acquires a signal value y(t+M) at time t+M as a prediction signal from the feature vector. For example, when the feature vector illustrated in FIG. 7 is acquired by the feature vector acquiring unit 130, the feature vector conversion unit 140 acquires a signal value y(t)+d(t+1)+ . . . +d(t+M) obtained by adding a sum of the elements d(t+1) to d(t+M) of the feature vector from time t+1 to time t+M to the signal value y(t) at time t as a signal value y(t+M) at time t+M. The feature vector conversion unit 140 outputs the calculated prediction signal to a monitor or the like of the time-series signal prediction device 100, and an operator of the treatment device 10 determines an imaging timing of an X-ray video or an irradiation timing of the treatment beam B while checking the prediction signal output to the monitor or the like and performs imaging of the X-ray video or irradiation with the treatment beam B.

The feature vector conversion unit 140 calculates a reliability of the time-series signal on the basis of the feature vector acquired by the feature vector acquiring unit 130. The feature vector conversion unit 140 calculates a smaller value as the reliability, for example, in case of a time-series signal in which the elements of the feature vector are discontinuous. Here, discontinuity can be determined, for example, using an approximate curve acquired from a plurality of elements included in the feature vector using a method such as spline interpolation and an approximation error between the plurality of elements. At this time, the feature vector conversion unit 140 may output the prediction signal and the calculated reliability to the monitor or the like of the time-series signal prediction device 100. In this case, an operator of the treatment device 10 can use the output prediction signal for imaging of the X-ray video or irradiation with the treatment beam B in consideration of the reliability of the prediction signal.

### [Process Flow]

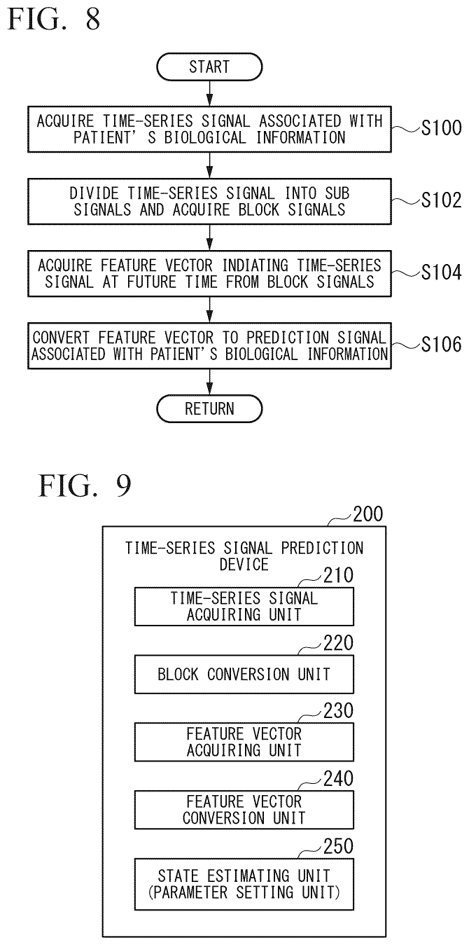

A process flow that is performed by the time-series signal prediction device according to the first embodiment will be described below with reference to FIG. 8. FIG. 8 is a flowchart illustrating an example of a process flow that is performed by the time-series signal prediction device according to the first embodiment.

First, the time-series signal acquiring unit 110 acquires a time-series signal associated with biological information of a patient P from the sensor S (Step S100). Then, the block conversion unit 120 divides the acquired time-series signal into sub signals and acquires the sub signals as block signals (Step S102). Then, the feature vector acquiring unit 130 acquires a feature vector indicating a time-series signal at a future time from the acquired block signals (Step S104). Then, the feature vector conversion unit converts the acquired feature vector to a prediction signal associated with the biological information of the patient P (Step S106). As a result, this process flow of the flowchart ends.

According to the first embodiment described above, an amount of information of input data is maintained and an input length is shortened by converting an input time-series signal to block signals. Accordingly, in comparison with the related art in which time-series signals are sequentially input, particularly, it is possible to shorten a time required for training of a recurrent neural network model and calculation of a feature vector. According to the first embodiment, in comparison with the technique described in Non Patent Document 1 in which only signal values to be predicted are learned in training of a model, an amount of information to be learned is increased by learning a plurality of signal values up to a time of prediction. Accordingly, it is possible to improve the accuracy of the feature vector output by the predictor.

### Second Embodiment

### (Entire Configuration)

In the first embodiment, the method of predicting a time-series signal at a future time from a time-series signal input in association with biological information of a patient P has been described above. Since an amount of air inhaled in inhalation varies even in the same patient P, the magnitude of the amplitude of a respiratory signal which is an example of biological information of a patient P when the patient P has inhaled up tends to become irregular. On the other hand, since the amplitude when the patient P has exhaled up is stable, the magnitude of the amplitude tends to become regular. In radiation treatment using this tendency, it is preferable to avoid capturing an X-ray video and irradiation with a treatment beam B during inhalation or exhalation of the patient P and to set a position of a tumor when the patient P has exhaled up as an irradiation spot. In consideration of these circumstances, a time-series signal prediction device 200 according to a second embodiment can predict a time at which a position of a tumor enters an irradiation spot and enable capturing of an X-ray video and irradiation with a treatment beam B to be avoided when the patient P is in inhalation or in exhalation or when a respiration state of the patient P is abnormal.

FIG. 9 is a block diagram schematically illustrating a configuration of the time-series signal prediction device 200 according to the second embodiment. The time-series signal prediction device 200 includes, for example, a time-series signal acquiring unit 210, a block conversion unit 220, a feature vector acquiring unit 230, a feature vector conversion unit 240, and a state estimating unit 250. The configurations of the time-series signal acquiring unit 210, the block conversion unit 220, the feature vector acquiring unit 230, and the feature vector conversion unit 240 are the same as the configurations of the time-series signal acquiring unit 110, the block conversion unit 120, the feature vector acquiring unit 130, and the feature vector conversion unit 140 of the time-series signal prediction device 100 according to the first embodiment, and thus description thereof will be omitted. In the second embodiment, the configuration of the treatment system 1 illustrated in FIG. 1 is employed in the same way.

The state estimating unit 250 estimates a state of a patient P on the basis of a prediction signal acquired from the feature vector conversion unit 240 and outputs the estimated state to a monitor or the like of the time-series signal prediction device 200. Here, the state of a patient to be estimated is, for example, a state (an abnormal state) departing from normal respiration due to coughs or the like. Before and after coughs or sneezes, respiration is disturbed and becomes different from a normal state, and thus a difference between a predicted value and a measured value increases. The state estimating unit 250 may output the estimated state of the patient to the monitor or the like of the treatment device 10.

For example, the state estimating unit 250 may calculate an absolute value of a difference between a predicted value acquired a predetermined time ago (for example, 100 ms ago) and a measured value at that time and estimate and output that the state of the patient is an abnormal state when the calculated absolute value is greater than a predetermined numerical value. For example, the state estimating unit 250 may calculate an absolute value of a difference between a predicted value acquired a predetermined time ago and a measured value at that time and estimate and output that the state of the patient is an abnormal state when a state in which the calculated absolute value is greater than a predetermined numerical value is maintained in a predetermined period. For example, the state estimating unit 250 may calculate an absolute value of a difference between a predicted value acquired a predetermined time ago and a measured value at that time using a plurality of time windows and estimate and output that the state of the patient is an abnormal state when the number of time windows in which the calculated absolute value is greater than a predetermined numerical value is major out of a plurality of time windows.

For example, the state of a patient may be a respiration phase state in the vicinity of a time at which respiration of the patient changes from exhalation to inhalation, that is, in the vicinity of a time at which the patient has exhaled up. When the amplitude of a respiratory waveform increases at the time of inhalation and decreases at the time of exhalation, the respiration enters the respiration phase state at a time at which the amplitude value is less than a certain threshold value. For example, this threshold value is set in advance from respiratory waveforms of the same patient corresponding to several respirations in the past by a user having monitored the respiratory waveform displayed on the monitor or the like of the time-series signal prediction device 200. For example, the user may set the threshold value to a predetermined level (for example, a 20% level) of the maximum amplitude in the time-series signal acquired by the time-series signal acquiring unit 110. The past is, for example, until set-up such as alignment of a patient has ended and treatment has started. Alternatively, the state estimating unit 250 may automatically identify the maximum amplitude in the time-series signal acquired by the time-series signal acquiring unit 110 and set the threshold value to a predetermined level of the identified maximum amplitude without causing a user to set the threshold value. In this case, the state estimating unit 250 has a function of a “parameter setting unit.”

When the prediction signal is a trajectory of a tracking target such as a tumor, a diaphragm, or a marker in a fluoroscopic video, the state estimating unit 250 may estimate and output the state of the tracking target in the fluoroscopic video. A tracking target such as a tumor, a diaphragm, or a marker in a fluoroscopic video may deteriorate in image quality and have difficulty tracking due to an increase in noise in the fluoroscopic image. A certain abnormality (for example, a cough or a sneeze) occurs in a patient P, and the tracking target may move irregularly. Accordingly, the state estimating unit 250 may estimate and output that the state of the tracking target is an abnormal state when the difference between a predicted value and a measured value of the prediction signal increases. For example, similarly to the state estimation associated with a respiratory waveform, the state estimating unit 250 may set a threshold value as described above when a tumor is planned to enter the irradiation spot in the vicinity of a time at which the amplitude of the time-series signal including the trajectory of a tracking target changes from down to up and estimate and output that the tumor is in the irradiation spot when the amplitude of the time-series signal is equal to or less than the threshold value.

It has been described above that the state estimating unit 250 is included in the time-series signal prediction device 200, but the state estimating unit 250 may be included in the treatment device 10. In this case, the time-series signal prediction device 200 may transmit the prediction signal acquired from the feature vector conversion unit 240 to the treatment device 10, and the state estimating unit 250 included in the treatment device 10 may estimate the state of the patient P on the basis of the received prediction signal and display the estimated state on the monitor or the like of the time-series signal prediction device 200.

### [Process Flow]

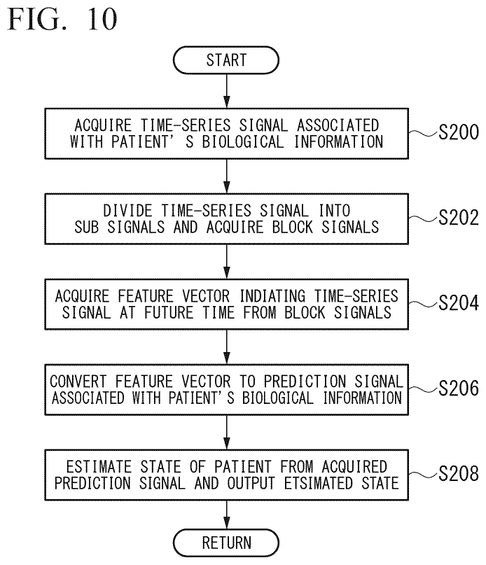

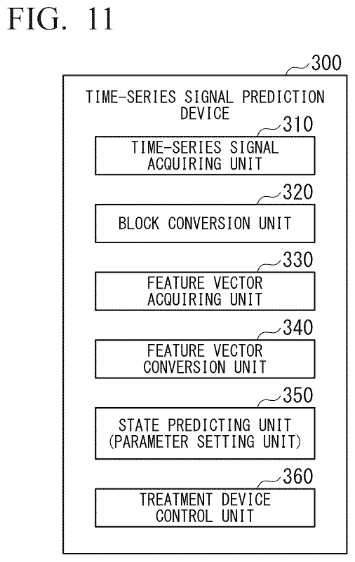

A process flow that is performed by the time-series signal prediction device according to the second embodiment will be described below with reference to FIG. 10. FIG. 10 is a flowchart illustrating an example of a process flow that is performed by the time-series signal prediction device according to the second embodiment.

First, the time-series signal acquiring unit 210 acquires a time-series signal associated with biological information of a patient P from the sensor S (Step S200). Then, the block conversion unit 220 divides the acquired time-series signal into sub signals and acquires the sub signals as block signals (Step S202). Then, the feature vector acquiring unit 230 acquires a feature vector indicating a time-series signal at a future time from the acquired block signals (Step S204). Then, the feature vector conversion unit 240 converts the acquired feature vector to a prediction signal associated with the biological information of the patient P (Step S206). Then, the state estimating unit 250 estimates a state of the patient P from the acquired prediction signal and outputs the estimated state to the monitor or the like of the time-series signal prediction device 200 (Step S208). As a result, this process flow of the flowchart ends.

According to the second embodiment described above, the state of the patient P is estimated from the prediction signal acquired in association with the biological information of the patient P and is output to the monitor or the like of the time-series signal prediction device 200, and, for example, an operator of the treatment device 10 determines an imaging timing of an X-ray video or an irradiation timing of a treatment beam while ascertaining the state of the patient P output to the monitor or the like and performs capturing of the X-ray video or irradiation with a treatment beam. Accordingly, it is possible to reduce unnecessary exposure accompanying imaging of an X-ray video or irradiation with a treatment beam.

### Third Embodiment

In the second embodiment, the method of estimating a state of a patient P from a prediction signal acquired in association with biological information of the patient P and outputting the estimated state to the monitor or the like of the time-series signal prediction device 200 has been described above. In this case, an operator of the treatment device 10 manually performs capturing an X-ray video and irradiation with a treatment beam B while ascertaining the state of the patient P output to the monitor or the like, and an artificial operation mistake or a miss in execution timing may occur. In consideration of these circumstances, a time-series signal prediction device 300 according to a third embodiment automatically controls the treatment device 10 on the basis of the estimated state of a patient P.

FIG. 11 is a block diagram schematically illustrating a configuration of the time-series signal prediction device 300 according to the third embodiment. The time-series signal prediction device 300 includes, for example, a time-series signal acquiring unit 310, a block conversion unit 320, a feature vector acquiring unit 330, a feature vector conversion unit 340, a state estimating unit 350, and a treatment device control unit 360. The configurations of the time-series signal acquiring unit 310, the block conversion unit 320, the feature vector acquiring unit 330, the feature vector conversion unit 340, and the state estimating unit 350 are the same as the configurations of the time-series signal acquiring unit 210, the block conversion unit 220, the feature vector acquiring unit 230, the feature vector conversion unit 240, and the state estimating unit 250 of the time-series signal prediction device 200 according to the second embodiment, and thus description thereof will be omitted. In the third embodiment, the configuration of the treatment system 1 illustrated in FIG. 1 is employed in the same way.

The treatment device control unit 360 controls the treatment device 10 on the basis of the state of the patient P estimated by the state estimating unit 350. More specifically, when the state estimating unit 350 estimates that the state of the patient P is a respiration phase in the vicinity of a time at which the patient has exhaled up (that is, when the amplitude value is less than a certain threshold value), the treatment device control unit 360 starts preparation of capturing of an X-ray video of the patient P and/or irradiation with a treatment beam B from the treatment device 10. For example, when the state estimating unit 350 estimates that a tumor in a fluoroscopic video is present in the irradiation spot, the treatment device control unit 360 may shorten an imaging interval of the X-ray video and start preparation of irradiation with a treatment beam B from the treatment device 10.

On the other hand, when the state estimating unit 350 determines that the state of the patient P is an abnormal state, the treatment device control unit 360 stops capturing of an X-ray video of the patient P and/or irradiation with a treatment beam B from the treatment device 10 or shortens an interval therebetween. For example, when the state estimating unit 350 estimates that the patient P has exhaled up and has transitioned to inhalation again, the treatment device control unit 360 may stop capturing of an X-ray video of the patient P and/or irradiation with a treatment beam B from the treatment device 10. For example, when the state estimating unit 350 estimates that a trajectory of a tracking target such as a tumor, a diaphragm, and a marker in a fluoroscopic video is in an abnormal state, the treatment device control unit 360 may stop capturing of an X-ray video of the patient P and/or irradiation with a treatment beam B from the treatment device 10.

It has been described above that the treatment device control unit 360 is included in the time-series signal prediction device 300, but the treatment device control unit 360 may be included in the treatment device 10. In this case, the time-series signal prediction device 300 may transmit the state of the patient P estimated by the state estimating unit 350 to the treatment device 10, and the treatment device control unit 360 included in the treatment device 10 may control the treatment device 10 on the basis of the received state of the patient P.

### [Process Flow]

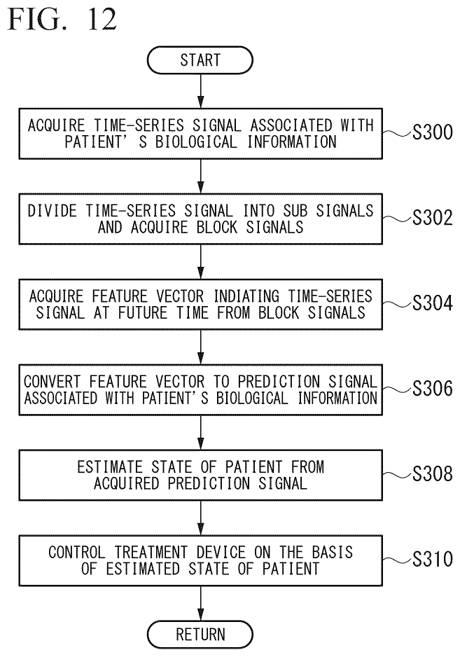

A process flow that is performed by the time-series signal prediction device 300 according to the third embodiment will be described below with reference to FIG. 12. FIG. 12 is a flowchart illustrating an example of a process flow that is performed by the time-series signal prediction device according to the third embodiment.

First, the time-series signal acquiring unit 310 acquires a time-series signal associated with biological information of a patient P from the sensor S (Step S300). Then, the block conversion unit 320 divides the acquired time-series signal into sub signals and acquires the sub signals as block signals (Step S302). Then, the feature vector acquiring unit 330 acquires a feature vector indicating a time-series signal at a future time from the acquired block signals (Step S304). Then, the feature vector conversion unit 340 converts the acquired feature vector to a prediction signal associated with the biological information of the patient P (Step S306). Then, the state estimating unit 350 estimates a state of the patient P from the acquired prediction signal (Step S308). Then, the treatment device control unit 360 controls the treatment device 10 on the basis of the estimated state of the patient P. As a result, this process flow of the flowchart ends.

According to the third embodiment described above, the treatment device 10 is controlled on the basis of the estimated state of the patient P such that capturing of an X-ray video of the patient P and/or irradiation with a treatment beam B from the treatment device 10 is performed or stopped. Accordingly, it is possible to further reduce unnecessary exposure accompanying capturing of an X-ray video or irradiation with a treatment beam.

While some embodiments of the present invention have been described above, these embodiments are provided as examples and are not intended to limit the scope of the present invention. These embodiments can be realized in various other forms, and various omissions, substitutions, and modifications can be added thereto without departing from the gist of the present invention. These embodiments and modifications thereof are included in the scope or gist of the present invention and are also included in the inventions described in the appended claims and equivalent scopes thereof.

The time-series signal prediction device, the radiation treatment device, the time-series signal prediction method, the program, and the storage medium according to the present invention employ the following configurations.

(1) A time-series signal prediction device according to an aspect of the present invention includes a time-series signal acquiring unit configured to acquire a time-series signal associated with a patient's biological information, a block conversion unit configured to divide the time-series signal into sub signals at predetermined intervals and to convert the sub signals to block signals of a time series which is shorter than a time series of the time-series signal, a feature vector acquiring unit configured to acquire, from the block signals, a feature vector indicating a time-series signal at a time subsequent to the time at which the time-series signal has been acquired, and a feature vector conversion unit configured to convert the feature vector to a prediction signal associated with the patient's biological information.

(2) In the aspect of (1), the time-series signal prediction device further includes a state estimating unit configured to estimate a state of the patient from the prediction signal.

(3) In the aspect of (2), the time-series signal prediction device further includes a parameter setting unit configured to adjust a parameter used for prediction in the state estimating unit on the basis of the time-series signal and the prediction signal.

(4) In the aspects of (1) to (3), the plurality of block signals include a signal at a certain time in duplicate.

(5) In the aspects of (1) to (4), the feature vector conversion unit calculates a reliability of the prediction signal from the feature vector.

(6) In the aspects of (1) to (5), the time-series signal is a signal varying with the patient's respiration, and the state is a state in which a respiratory motion of the patient is abnormal.

(7) In the aspects of (1) to (5), the time-series signal is a signal varying with the patient's respiration, and the state is a timing at which a respiratory motion of the patient changes from exhalation to inhalation.

(8) In the aspects of (1) to (5), the time-series signal is a signal varying according to a tumor position of the patient, and the prediction signal is a predicted position of a tumor of the patient.

(9) A radiation treatment device according to another aspect of the present invention controls an imaging timing of a fluoroscopic image of the patient on the basis of the state predicted by the time-series signal prediction device according to the aspect of (2).

(10) A radiation treatment device according to another aspect of the present invention controls an irradiation timing of the patient with a treatment beam on the basis of the state predicted by the time-series signal prediction device according to the aspect of (2).

(11) A time-series signal prediction method according to another aspect of the present invention is performed by a computer and includes acquiring a time-series signal associated with a patient's biological information, dividing the time-series signal into sub signals at predetermined intervals and converting the sub signals to block signals of a time series which is shorter than a time series of the time-series signal, acquiring, from the block signals, a feature vector indicating a time-series signal at a time subsequent to the time at which the time-series signal has been acquired, and converting the feature vector to a prediction signal associated with the patient's biological information.

(12) A program according to another aspect of the present invention causes a computer to perform acquiring a time-series signal associated with a patient's biological information, dividing the time-series signal into sub signals at predetermined intervals and converting the sub signals to block signals of a time series which is shorter than a time series of the time-series signal, acquiring, from the block signals, a feature vector indicating a time-series signal at a time subsequent to the time at which the time-series signal has been acquired, and converting the feature vector to a prediction signal associated with the patient's biological information.

(13) A storage medium according to another aspect of the present invention stores a program causing a computer to perform acquiring a time-series signal associated with a patient's biological information, dividing the time-series signal into sub signals at predetermined intervals and converting the sub signals to block signals of a time series which is shorter than a time series of the time-series signal, acquiring, from the block signals, a feature vector indicating a time-series signal at a time subsequent to the time at which the time-series signal has been acquired, and converting the feature vector to a prediction signal associated with the patient's biological information.

What is claimed is:

1. A time-series signal prediction device comprising:
a time-series signal acquirer configured to acquire a time-series signal associated with a patient's biological information;
a block converter configured to divide the time-series signal into sub signals at predetermined intervals and to convert the sub signals to block signals of a time series which is shorter than a time series of the time-series signal;
a feature vector acquirer configured to acquire, from the block signals, a feature vector indicating a time-series signal at a time subsequent to the time at which the time-series signal has been acquired; and
a feature vector converter configured to convert the feature vector to a prediction signal associated with the patient's biological information.

2. The time-series signal prediction device according to claim 1, further comprising a state estimator configured to estimate a state of the patient from the prediction signal.

3. The time-series signal prediction device according to claim 2, further comprising a parameter setter configured to adjust a parameter used for prediction in the state estimator on the basis of the time-series signal and the prediction signal.

4. The time-series signal prediction device according to claim 1, wherein the plurality of block signals include a signal at a certain time in duplicate.

5. The time-series signal prediction device according to claim 1, wherein the feature vector converter calculates a reliability of the prediction signal from the feature vector.

6. The time-series signal prediction device according to claim 2, wherein the time-series signal is a signal varying with the patient's respiration, and the state is a state in which a respiratory motion of the patient is abnormal.

7. The time-series signal prediction device according to claim 2, wherein the time-series signal is a signal varying with the patient's respiration, and the state is a timing at which a respiratory motion of the patient changes from exhalation to inhalation.

8. The time-series signal prediction device according to claim 1, wherein the time-series signal is a signal varying according to a tumor position of the patient, and the prediction signal is a predicted position of a tumor of the patient.

9. A radiation treatment device that controls an imaging timing of a fluoroscopic image of the patient on the basis of the state predicted by the time-series signal prediction device according to claim 2.

10. A radiation treatment device that controls an irradiation timing of the patient with a treatment beam on the basis of the state predicted by the time-series signal prediction device according to claim 2.

11. A time-series signal prediction method that is performed by a computer, the time-series signal prediction method comprising:
acquiring a time-series signal associated with a patient's biological information;
dividing the time-series signal into sub signals at predetermined intervals and converting the sub signals to block signals of a time series which is shorter than a time series of the time-series signal;
acquiring, from the block signals, a feature vector indicating a time-series signal at a time subsequent to the time at which the time-series signal has been acquired; and
converting the feature vector to a prediction signal associated with the patient's biological information.

12. A non-transitory computer-readable storage medium storing a program causing a computer to perform:
acquiring a time-series signal associated with a patient's biological information;
dividing the time-series signal into sub signals at predetermined intervals and converting the sub signals to block signals of a time series which is shorter than a time series of the time-series signal;
acquiring, from the block signals, a feature vector indicating a time-series signal at a time subsequent to the time at which the time-series signal has been acquired; and
converting the feature vector to a prediction signal associated with the patient's biological information.
